(19) United States
(12) Patent Application Publication
(10) Pub. No.: US 2019/0338356 A1
(43) Pub. Date: Nov. 7, 2019

(54) CONSTRUCTS AND METHODS FOR SIGNAL AMPLIFICATION

(71) Applicant: THE REGENTS OF THE UNIVERSITY OF CALIFORNIA, Oakland, CA (US)

(72) Inventors: Paul A. Feldstein, Sacramento, CA (US); Marc Pollack, Davis, CA (US); Bryce Falk, Davis, CA (US)

(73) Assignee: THE REGENTS OF THE UNIVERSITY OF CALIFORNIA, Oakland, CA (US)

(21) Appl. No.: 16/270,198

(22) Filed: Feb. 7, 2019

Related U.S. Application Data
(63) Continuation of application No. PCT/US2017/046260, filed on Aug. 10, 2017.
(60) Provisional application No. 62/374,746, filed on Aug. 12, 2016.

(51) Int. Cl. C12Q 1/6876; C12Q 1/6832

(52) U.S. Cl. CPC C12Q 1/6876; C12Q 1/6832

(57) ABSTRACT

Compositions and methods for the detection of rare target polynucleotides in a complex mixture.

### CROSS-REFERENCE TO RELATED APPLICATIONS

This application claims priority to, and is a 35 U.S.C. § 111(a) continuation of, PCT international application number PCT/US2017/046260 filed on Aug. 10, 2017, incorporated herein by reference in its entirety, which claims priority to, and the benefit of, U.S. provisional patent application Ser. No. 62/374,746 filed on Aug. 12, 2016, incorporated herein by reference in its entirety. Priority is claimed to each of the foregoing applications.

The above-referenced PCT international application was published as PCT International Publication No. WO 2018/031751 A1 on Feb. 15, 2018, which publication is incorporated herein by reference in its entirety.

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

Current methodologies for the detection of the rare polynucleotides of an organism in a complex mixture require either detection of the DNA of the organism through methods such as polymerase chain reaction, which can amplify the rare nucleotides to levels where detection is possible, or detection of the RNA of the organism using methods such as reverse transcriptase-polymerase chain reaction, which suffers from the same fundamental problems. Such methods require expensive reagents and complex machinery just for amplification after which a separate detection system is necessary to ensure that the amplified nucleic acid is in fact from the organism to be detected. Also, if the organism is present in a few or even one copy, its DNA is likely to be present in only a few or one copy making detection even more difficult.

RNA-based detection has the advantage of a natural amplification through transcription, the cellular process of converting DNA to RNA as part of the information transfer in cells. While a single cell of an organism to be detected may contain only a single copy of DNA, it is likely to contain tens, hundreds or even thousands or copies of the RNA transcribed from that DNA. Unfortunately, the inherently greater instability of RNA makes its handling more difficult to ensure that the RNA is sufficiently intact to allow successful detection. If the RNA is degraded such that the average size of the RNA recovered is close to or smaller than the distance between the two sequences necessary for most amplification methodologies, the detection efficiency and ability to correctly quantify the amount of RNA present will suffer. Also, methods for detecting RNA require the use of extra reagents, such as reverse transcriptase, an enzyme that converts RNA to complementary DNA, before downstream amplification and detection are possible. This leads to an increase in cost. Also, because the amplification must be sequence-specific to allow the organism's nucleic acid to be selectively amplified out of the complex mixtures of nucleic acids found in a biological sample, each amplified nucleic acid will have a unique sequence making downstream detection more complex.

### SUMMARY

In one aspect, provided are translator DNA constructs. In varying embodiments, the translator DNA constructs comprise the following operably linked polynucleotide elements in the 5′ to 3′ direction:

a) a transcriptional hairpin portion, comprising:

i) a first detection sequence in the antisense orientation;

ii) a first RNA polymerase promoter sequence in the antisense orientation;

iii) a hairpin linker;

iv) the first RNA polymerase promoter sequence in the sense orientation; and

v) the first detection sequence in the sense orientation;

b) a flexible linker; and

c) a recognition sequence that hybridizes to a target sequence.

In varying embodiments, the translator DNA constructs comprise the following operably linked polynucleotide elements in the 5′ to 3′ direction:

a) a recognition sequence that hybridizes to a target sequence

b) a flexible linker; and

c) a transcriptional hairpin portion, comprising:

i) a first detection sequence in the antisense orientation;

ii) a first RNA polymerase promoter sequence in the antisense orientation;

iii) a hairpin linker;

iv) the first RNA polymerase promoter sequence in the sense orientation; and

v) the first detection sequence in the sense orientation. In further varying embodiments of the translator constructs, the first detection sequence in the sense orientation can be transcribed into RNA from the promoter, producing an RNA oligonucleotide comprising the first detection sequence in the sense orientation. In varying embodiments, the first promoter is functional in a prokaryotic cell. In varying embodiments, the first promoter functional in a prokaryotic cell comprises a bacteriophage promoter selected from the group consisting of T7, T3 and SP6. In varying embodiments, the translator DNA construct has an overall length in the range of about 50 bp to about 150 bp, e.g., from about 60 bp to about 140 bp, e.g., from about 65 bp to about 130 bp, e.g., from about 69 bp to about 129 bp, e.g., about 50, 55, 60, 65, 70, 75, 80, 85, 90, 95, 100, 105, 110, 115, 120, 125, 130, 135, 140, 145, 150 bp. In varying embodiments, the recognition sequence has a length in the range of about 10 bp to about 50 bp, e.g, from about 10 bp to about 40 bp, e.g., about 10 bp to about 30 bp, e.g., about 10, 15, 20, 25, 30, 35, 40, 45, 50 bp. In varying embodiments, the first detection sequence has a length in the range of about 10 bp to about 50 bp, e.g, from about 10 bp to about 40 bp, e.g., about 10 bp to about 30 bp, e.g., about 10, 15, 20, 25, 30, 35, 40, 45, 50 bp. In varying embodiments, the hairpin linker has a length in the range of about 4 bp to about 10 bp, about 4, 5, 6, 7, 8, 9, 10 bp. In varying embodiments, the hairpin linker comprises a polyethylene glycol linker, e.g., having 3, 6, 9, 12, 15, or 18 carbons. In varying embodiments, the hairpin linker comprises a deoxyribose phosphodiester linker without nucleotide bases. In varying embodiments, the flexible linker comprises a polyethylene glycol linker, e.g., having 3, 6, 9, 12, 15, or 18 carbons. In varying embodiments, the flexible linker comprises a deoxyribose phosphodiester linker without nucleotide bases. In varying embodiments, the translator DNA construct is attached to a solid support.

In another aspect, provided is an amplifier DNA construct. In varying embodiments, the amplifier DNA construct comprises the following operably linked polynucleotide elements in the 5′ to 3′ direction:

a) a second detection sequence in the antisense orientation;

b) a second RNA polymerase promoter in the antisense orientation; and

c) the first detection sequence in the antisense orientation, as described above and herein. In varying embodiments, the first detection sequence and the second detection sequence are the same. In varying embodiments, the first detection sequence and the second detection sequence are different. In varying embodiments, the RNA oligonucleotide comprising the first detection sequence in the sense orientation produced from the translator DNA construct, as described above and herein, anneals or hybridizes to the first detection sequence in the antisense orientation of the amplifier DNA construct, allowing for extension of the RNA oligonucleotide into the promoter and the second detection sequence in the sense orientation from the amplifier construct, and further the transcription of the second detection sequence in the sense orientation, producing an RNA oligonucleotide comprising the second detection sequence in the sense orientation. In varying embodiments, the second promoter is functional in a prokaryotic cell. In varying embodiments, the second promoter functional in a prokaryotic cell comprises a bacteriophage promoter selected from the group consisting of T7, T3 and SP6. In varying embodiments, the first promoter and the second promoter are the same. In varying embodiments, the first promoter and the second promoter are different. In varying embodiments, the amplifier DNA construct has an overall length in the range of about 35 bp to about 80 bp, e.g., from about 37 bp to about 77 bp, e.g., about 35, 40, 45, 50, 55, 60, 65, 70, 75, 80 bp. In varying embodiments, the second detection sequence has a length in the range of about 10 bp to about 50 bp, e.g, from about 10 bp to about 40 bp, e.g., about 10 bp to about 30 bp, e.g., about 10, 15, 20, 25, 30, 35, 40, 45, 50 bp. In varying embodiments, the amplifier DNA construct is attached to a solid support.

In a further aspect, provided is an amplifier DNA construct. In some embodiments, the amplifier DNA construct comprises the following operably linked polynucleotide elements in the 5′ to 3′ direction:

a) a second promoter in the sense orientation;

b) a second detection sequence in the sense orientation;

c) a synthetic transcription termination sequence; and

d) the first detection sequence in the antisense orientation, as described above and herein. In varying embodiments, the first detection sequence and the second detection sequence are the same. In varying embodiments, the first detection sequence and the second detection sequence are different. In varying embodiments, the RNA oligonucleotide comprising the first detection sequence in the sense orientation produced from the translator DNA construct, as described above and herein, anneals or hybridizes to at least the first detection sequence in the antisense orientation of the amplifier DNA construct, allowing for extension of the RNA oligonucleotide into the transcription termination sequence, the second detection sequence and the promoter in the antisense orientation from the amplifier construct, and further the transcription of the second detection sequence in the sense orientation, producing an RNA oligonucleotide comprising the second detection sequence in the sense orientation. In varying embodiments, the promoter is functional in a prokaryotic cell. In varying embodiments, the first promoter and the second promoter are the same. In varying embodiments, the first promoter and the second promoter are different. In varying embodiments, the promoter functional in a prokaryotic cell comprises a bacteriophage promoter selected from the group consisting of T7, T3 and SP6. In varying embodiments, the amplifier DNA construct has an overall length in the range of about 35 bp to about 85 bp, e.g., from about 38 bp to about 82 bp, e.g., about 35, 40, 45, 50, 55, 60, 65, 70, 75, 80, 85 bp. In varying embodiments, the second detection sequence has a length in the range of about 10 bp to about 50 bp, e.g, from about 10 bp to about 40 bp, e.g., about 10 bp to about 30 bp, e.g., about 10, 15, 20, 25, 30, 35, 40, 45, 50 bp. In varying embodiments, the synthetic transcription termination sequence has a length in the range of about 1 bp to about 5 bp, e.g., about 1, 2, 3, 4, 5 bp. In varying embodiments, the amplifier DNA construct is attached to a solid support.

In a further aspect, provided are reaction mixtures comprising a translator DNA construct, as described above and herein. In varying embodiments, the reaction mixtures further comprise an amplifier DNA construct, as described above and herein. In varying embodiments, the reaction mixture comprises the plurality of translator DNA constructs at a concentration of about 10⁻³ M, about 10⁻⁴ M, about 10⁻⁵ M, about 10⁻⁶ M, about 10⁻⁷ M, about 10⁻⁸ M, about 10⁻⁹ M, about 10⁻¹¹ M, about 10⁻¹² M, or less. In varying embodiment, the molar ratio of the plurality of amplifier DNA constructs to the translator DNA constructs is at least about 2:1, e.g., from about 2:1 to about 10³:1, about 10⁴:1, about 10⁵:1, about 10⁶:1, or more.

In a further aspect, provided are kits comprising a translator DNA construct, as described above and herein. In varying embodiments, the kits further comprise an amplifier DNA construct, as described above and herein.

In a further aspect, provided are solid supports comprising a plurality of translator DNA constructs, as described above and herein, attached to the solid support at predetermined addressable locations. In another aspect, provided are solid supports comprising a plurality of amplifier DNA constructs, as described above and herein, attached to the solid support at predetermined addressable locations.

In another aspect, provided are methods of detecting the presence of a target polynucleotide sequence. In some embodiments, the methods comprise:

a) contacting a sample suspected of comprising the target polynucleotide sequence with a translator DNA construct, as described above and herein, under conditions that allow the translator DNA construct to anneal or hybridize to the target polynucleotide; thereby yielding a mixture of annealed or hybridized translator DNA constructs and unannealed or unhybridized translator DNA constructs;

b) separating annealed or hybridized translator DNA construct and target polynucleotide from unannealed or unhybridized translator DNA construct and target polynucleotide;

c) transcribing the first detection sequence, thereby producing an RNA oligonucleotide comprising the first detection sequence in the sense orientation; and

d) detecting the RNA oligonucleotide comprising the first detection sequence in the sense orientation, whereby detecting the RNA oligonucleotide transcribed from the first detection sequence indicates the presence of the target polynucleotide.

In some embodiments, the methods comprise:

a) contacting a sample suspected of comprising the target polynucleotide sequence with a translator DNA construct, as described above and herein, under conditions that allow the translator DNA construct to anneal or hybridize to the target polynucleotide; thereby yielding a mixture of annealed or hybridized translator DNA constructs and unannealed or unhybridized translator DNA constructs;

b) separating annealed or hybridized translator DNA construct and target polynucleotide from unannealed or unhybridized translator DNA construct and target polynucleotide;

c) transcribing the first detection sequence from the translator DNA construct, thereby producing an RNA oligonucleotide comprising the first detection sequence in the sense orientation;

d) contacting the RNA oligonucleotide comprising the first detection sequence in the sense orientation with an amplifier DNA construct, as described above and herein, under conditions that allow the RNA oligonucleotide comprising the first detection sequence in the sense orientation and the amplifier DNA construct to hybridize;

e) extending the RNA oligonucleotide comprising the first detection sequence in the sense orientation with a DNA polymerase thereby producing an extended DNA sequence comprising as operably linked polynucleotide elements in the 5′ to 3′ direction the second promoter and the second detection sequence; and

f) transcribing the second detection sequence from the extended DNA sequence, thereby producing an RNA oligonucleotide comprising the second detection sequence in the sense orientation, whereby detecting the RNA oligonucleotide transcribed from the second detection sequence indicates the presence of the target polynucleotide. In some embodiments, the target polynucleotide is RNA. In some embodiments, the target polynucleotide is DNA. In varying embodiments, the target polynucleotide is a DNA:RNA hybrid molecule. In some embodiments, the annealed or hybridized translator DNA construct with RNA target polynucleotide is stabilized by a mutated RNase H that does not have nuclease activity. In varying embodiments, one of the translator DNA construct or the target polynucleotide is attached to a solid support. In varying embodiments, the sample is selected from the group consisting of a food sample, a biological sample, a water sample or a soil sample. In varying embodiments, the RNA oligonucleotide is detected by a method selected from the group consisting of radioactive nucleotide incorporation; fluorescent nucleotide incorporation; chemically-derivatized nucleotide incorporation followed by radioactive, fluorescent or enzymatic activity detection; electrochemical detection, molecular conductance detection; detection of the formation of double stranded DNA; detection of transcription of an inherently fluorescent aptamer; detection of transcription of an aptamer that is fluorescent after binding of an otherwise non-fluorescent or minimally-fluorescent molecule; and detection of pyrophosphate produced by transcription and DNA polymerase activity. In varying embodiments, the sample comprises a plurality of different target polynucleotides and a plurality of different translator DNA constructs. In varying embodiments, as few as one target polynucleotide is, e.g., as few as 2, 3, 4, 5, 6, 7, 8, 9, 10, 11, 12, 13, 14, 15, 16, 17, 18, 19, 20 target polynucleotides are, present in the sample.

### Definitions

The term “plurality” refers to two or more. As appropriate, a “plurality” can refer to 2, 4, 8, 16, 32, 64, 100, 500, 1000, or more.

### BRIEF DESCRIPTION OF THE DRAWINGS

FIG. 1 illustrates a structure of a translator oligonucleotide. In varying embodiments, the linker can be a flexible attachment between the recognition sequence and the transcription portion (e.g., a hairpin linker comprising a promoter operably linked to a first detection sequence in the sense orientation) of the translator oligonucleotide and give the two regions freedom to move relative to each other. The 3′ phosphate prevents extension of the translator oligo when used in conjunction with amplifier oligonucleotides. “Sequence 1” refers to the first detection sequence in the sense orientation; “Sequence 1′” refers to the first detection sequence in the antisense orientation; and “Sequence 1 RNA” refers to the RNA transcribed from the first detection sequence in the sense orientation operably linked to the promoter in the sense orientation.

FIG. 2 illustrates a variant structure for a translator oligonucleotide. In this variant, the recognition sequence is linked by a linker acting as a flexible attachment between the 3′ end of the recognition sequence and the 5′ end of the transcription portion as opposed to the linker attachment being between the 3′ end of the transcription portion and the 5′ end of the recognition sequence as shown in FIG. 1.

FIGS. 3A-D illustrate the design and function of an amplifier oligonucleotide. DNA sequences are shown as solid lines. RNA sequences are shown as dashed lines. The structure of an embodiment of an amplifier oligonucleotide is depicted in line A. Its utilization is depicted in lines B-D. The promoter sequence in the antisense orientation (−) represents the complementary or negative strand of a promoter. The extension step is performed using a DNA polymerase.

FIGS. 4A-D illustrate the design and function of a cascade amplifier oligonucleotide. DNA sequences are shown as solid lines. RNA sequences are shown as dashed lines. The structure of an embodiment of an amplifier oligonucleotide is depicted in line A. Its utilization is depicted in lines B-D. The promoter sequence in the antisense orientation (−) represents the complementary or negative strand of a promoter. The extension step is performed using a DNA polymerase. The black arrow represents the feed forward binding and activation of the cascade amplifier by its own transcript.

FIGS. 5A-D illustrate the binding of Sequence 1 RNA to both repeats in a cascade amplifier oligonucleotide. DNA sequences are shown as solid lines. RNA sequences are shown as dashed lines. The structure of an embodiment of an amplifier oligonucleotide is depicted in line A. Its utilization is depicted in lines B-D. The promoter sequence in the antisense orientation (−) represents the complementary or negative strand of a promoter. The extension step is performed using a DNA polymerase. The black arrow represents the feed forward binding and activation of the next generation cascade amplifier by its own transcript.

FIGS. 6A-D illustrate the structure and function of an amplifier oligonucleotide. DNAs are shown as solid lines and RNAs are shown as dashed lines. The structure of an embodiment of an amplifier oligonucleotide is depicted in line A. Its utilization is depicted in lines B-D. From 5′ to 3′, it comprises a polymerase promoter; a second detection sequence (Sequence 2), which is synthesized from the second promoter; a stop sequence, (e.g., a small run of dA residues or a small run of d-isoC or d-isoG residues), which terminates the transcription of the second detection sequence; and the complement of the first detection sequence (Sequence 1) synthesized by the translator oligonucleotide in FIG. 1. Utilization of this embodiment of an amplifier oligonucleotide is depicted in lines B-D.

FIG. 7 illustrates possible internal secondary structure formed by embodiment of a cascade amplifier of the type depicted in FIG. 6.

FIG. 8 illustrates an alternative intramolecular RNA secondary structure that allows exposure of the 3′ binding site of “Sequence 1 RNA” (e.g., transcribed first detection sequence). The linker (shown as a long dashed line) holds the RNA and DNA strands together to ensure that the formation would be governed by intramolecular kinetics. The RNA sequence can be extended beyond the RNA promoter to include the stop sequences to help offset the lower stability of the DNA:RNA hybrid compared to the DNA:DNA hybrid as depicted in FIG. 9A. Mutant RNase H can be used to stabilize the desired DNA:RNA hybrid, especially if a high temperature RNase H, e.g. from Thermus thermophiles, is used. If the mutant RNase H is immobilized, only the correct hybrids are retained.

FIGS. 9A-B illustrate alternative intramolecular DNA:RNA or DNA:DNA secondary structures that would allow exposure of the 3′ binding site of “1 RNA” (e.g., transcribed first detection sequence) of a cascade amplifier of the type shown in FIG. 6. The structure of the cascade amplifier with intramolecular DNA:RNA secondary structure is depicted in line A. The linker (shown as a long dashed line) would serve to hold the DNA and RNA strands together to ensure that the formation would be governed by intramolecular kinetics. The RNA sequence would be extended into the RNA polymerase promoter and/or stop sequences to tip the thermodynamic balance towards the desired DNA:RNA hybrid. The use of mutant RNase H binding was used in this embodiment to ensure formation of the correct secondary structure or to aid in separation of the correct form from the incorrect form. The structure of the cascade amplifier with intramolecular DNA:DNA secondary structure is depicted in line B. The linker (shown as a long dashed line) would serve to hold the two DNA strands together to ensure that the formation would be governed by intramolecular kinetics. The DNA sequence would be extended into the RNA polymerase promoter and/or stop sequences to tip the thermodynamic balance towards the desired DNA:DNA hybrid. The use of an extra sequence (extra) between the stop and sequence 1′ that contains a DNA binding protein motif (e.g., for lambda cro or cl dimer proteins) is used analogously to how mutant RNase H was used in the embodiment depicted in line A to ensure formation of the correct secondary structure or to aid in separation of the correct form from the incorrect form.

FIG. 10 illustrates detection of an RNA molecule using a translator oligonucleotide. The RNA being detected is shown as a dashed line, while the capture DNA oligonucleotide (oligo 1) and the translator oligonucleotide are shown as solid lines. A mutant RNase H which binds and stabilizes DNA:RNA hybridized sequences, but which has reduced or eliminated RNase activity optionally can be used to stabilize DNA:RNA complexes.

FIG. 11 illustrates a ligation-based detection system using hybridization of an anchored DNA strand and a translator oligonucleotide hybridized to an RNA such that there is a juxtaposition of the ligatable DNA termini (5′ phosphate and 3′ hydroxyl) leading to a covalent attachment of the translator oligonucleotide to the surface followed by detection. Optional stabilization by binding of mutant RNase H can be used.

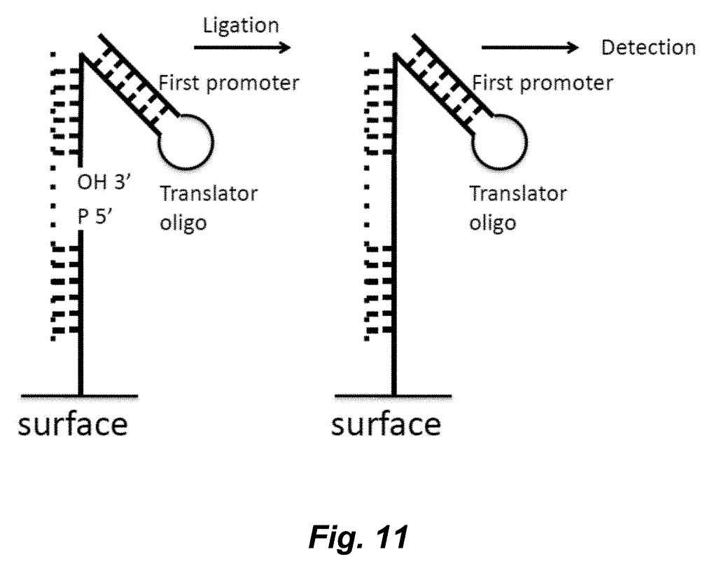

FIG. 12 illustrates a physical arrangement of amplifiers to increase signal strength. Detection can be at any or all amplification steps and can be using any of a number of detection technologies available. The detection methods described herein have the capability to detect a single RNA molecule from a complex mixture.

FIG. 13 illustrates use of mutant RNase H as an antenna for detecting DNA:RNA hybrid formation on a planar or nanoporous (np Au) surface. The DNA sequence (solid line) is attached to the surface and the RNA sequence (dashed line) is bound to the attached DNA sequence. Examples of derivative moieties that can be used include methylene blue and horseradish peroxidase (HRP), the enzymatic product of which can be detected electrochemically.

FIG. 14 illustrates isolation/purification of a specific RNA by selective binding of a DNA:RNA hybrid region. The RNA (dashed line) is bound by a capture DNA (solid line), which are then selectively bound by an immobilized mutant RNase H protein.

FIG. 15 illustrates detection of specific RNA by RT-PCR after capture of DNA:RNA hybrid. The RNA is shown as a dashed line and the cDNA being amplified as well as the primers used for the RT-PCR are shown as solid lines.

FIG. 16 illustrates detection of a specific RNA sequence using a second DNA oligonucleotide and a derivatized mutant RNase H protein. The RNA being detected is shown as a dashed line and the capture DNA oligonucleotide (oligo 1) and the second DNA oligonucleotide (oligo 2) are shown as solid lines.

FIG. 17 depicts a non-denaturing urea gel demonstrating the detection of a RNA:DNA hybrid molecule using a mutant RNase H according to the methods described in Example 1 and herein.

FIG. 18 depicts a denaturing urea gel demonstrating the detection of a RNA:DNA hybrid molecule using a mutant RNase H according to the methods described in Example 1 and herein.

FIG. 19 depicts a denaturing urea gel demonstrating transcription of a dilution series of translator oligonucleotides leading to activation of an amplifier oligonucleotide. See, Example 2.

### DETAILED DESCRIPTION

1. Introduction

Detection assays of high sensitivity and rapidity are provided that address a number of problems where either great sensitivity (down to a single molecule in some cases) or a rapid response to a novel pathogen is required. Illustrative applications include agriculture, where pathogens can be detected before they become widespread; in food safety, where small numbers of pathogenic bacteria can be detected before they enter the food supply; and medicine, where sepsis-causing agents can be detected early enough to change the clinical outcome for the patient.

2. Translator Constructs

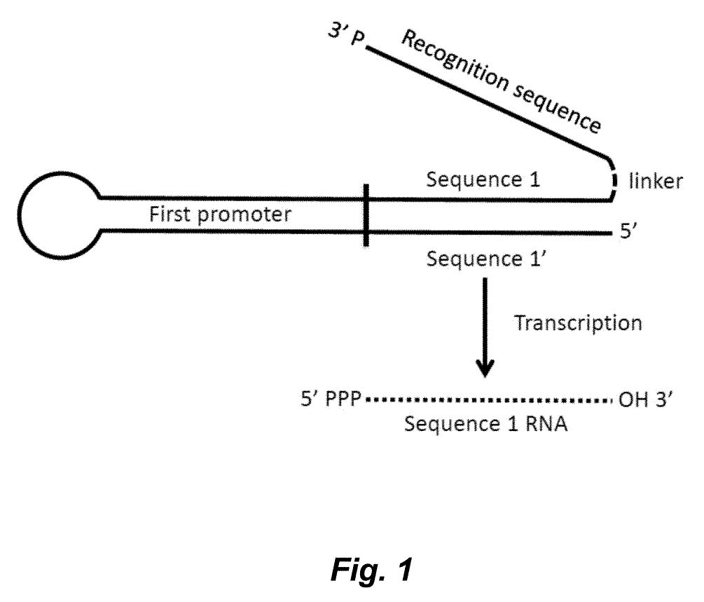

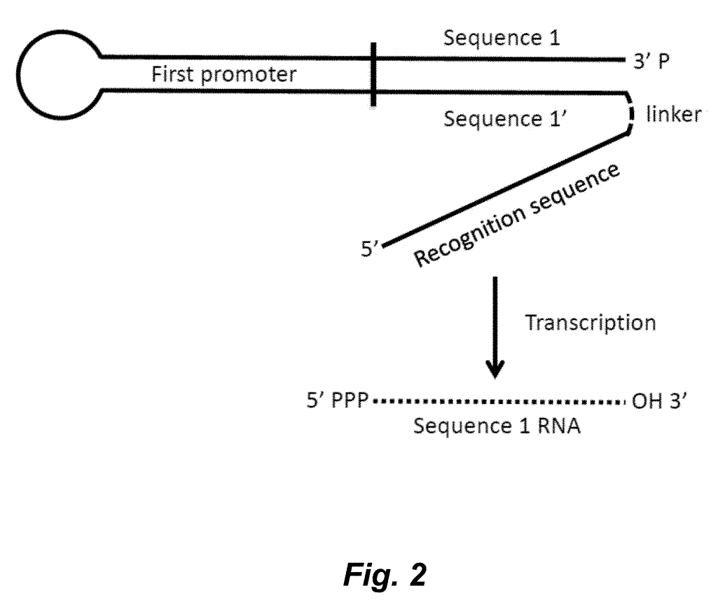

Provided are translator constructs. Illustrative structures of embodiments of translator oligonucleotides are depicted in FIGS. 1 and 2. Translator oligonucleotides generally have two regions linked by a flexible linker-(1) the recognition sequence, which is responsible for a specific interaction with the target RNA, e.g., mediated by normal Watson-Crick hydrogen bonding, which optionally can be stabilized by binding by a mutant RNase H protein, and (2) the transcription region, which transcribes a new RNA using an RNA polymerase promoter.

Accordingly, in varying embodiments, translator DNA constructs comprise the following operably linked polynucleotide elements in the 5′ to 3′ direction:

a) a transcriptional hairpin portion, comprising:

i) a first detection sequence in the antisense orientation;

ii) a first RNA polymerase promoter sequence in the antisense orientation;

iii) a hairpin linker;

iv) the first RNA polymerase promoter sequence in the sense orientation; and

v) the first detection sequence in the sense orientation;

b) a flexible linker; and

c) a recognition sequence that hybridizes to a target sequence.

In varying embodiments, the translator DNA constructs comprise the following operably linked polynucleotide elements in the 5′ to 3′ direction:

a) a recognition sequence that hybridizes to a target sequence

b) a flexible linker; and

c) a transcriptional hairpin portion, comprising:

i) a first detection sequence in the antisense orientation;

ii) a first RNA polymerase promoter sequence in the antisense orientation;

iii) a hairpin linker;

iv) the first RNA polymerase promoter sequence in the sense orientation; and

v) the first detection sequence in the sense orientation.

In varying embodiments, the first detection sequence in the sense orientation can be transcribed into RNA from the promoter, producing an RNA oligonucleotide comprising the first detection sequence in the sense orientation. In varying embodiments, the first promoter is functional in a prokaryotic cell. In varying embodiments, the first promoter functional in a prokaryotic cell comprises a bacteriophage promoter selected from the group consisting of T7, T3 and SP6. In varying embodiments, the translator DNA construct has an overall length in the range of about 50 bp to about 150 bp, e.g., from about 60 bp to about 140 bp, e.g., from about 65 bp to about 130 bp, e.g., from about 69 bp to about 129 bp, e.g., about 50, 55, 60, 65, 70, 75, 80, 85, 90, 95, 100, 105, 110, 115, 120, 125, 130, 135, 140, 145, 150 bp. In varying embodiments, the recognition sequence has a length in the range of about 10 bp to about 50 bp, e.g, from about 10 bp to about 40 bp, e.g., about 10 bp to about 30 bp, e.g., about 10, 15, 20, 25, 30, 35, 40, 45, 50 bp. In varying embodiments, the first detection sequence has a length in the range of about 10 bp to about 50 bp, e.g, from about 10 bp to about 40 bp, e.g., about 10 bp to about 30 bp, e.g., about 10, 15, 20, 25, 30, 35, 40, 45, 50 bp. In varying embodiments, the hairpin linker has a length in the range of about 4 bp to about 10 bp, about 4, 5, 6, 7, 8, 9, 10 bp. In varying embodiments, the hairpin linker comprises a polyethylene glycol linker, e.g., having 3, 6, 9, 12, 15, or 18 carbons. In varying embodiments, the hairpin linker comprises a deoxyribose phosphodiester linker without nucleotide bases. In varying embodiments, the flexible linker comprises a polyethylene glycol linker, e.g., having 3, 6, 9, 12, 15, or 18 carbons. In varying embodiments, the flexible linker comprises a deoxyribose phosphodiester linker without nucleotide bases. In varying embodiments, the translator DNA construct is attached to a solid support. The hairpin linker can have the same chemical structure as the flexible linker, but the flexible linker generally does not have normal nucleotides as the hairpin linker can.

The value in translator oligonucleotides is the ability to convert a specific binding event into the production of a new, specific RNA that can be detected by a downstream detector along with the natural amplification caused by transcription. The sequence of the new, specific RNA can be optimized to work in the downstream detector and be easily differentiated from other RNAs present, for example, other specific RNAs produced by other translator RNAs recognizing the same RNA. Once these optimized RNA sequences are determined, simply changing the recognition sequences will allow a change in the RNA being detected without the need to change the downstream detector.

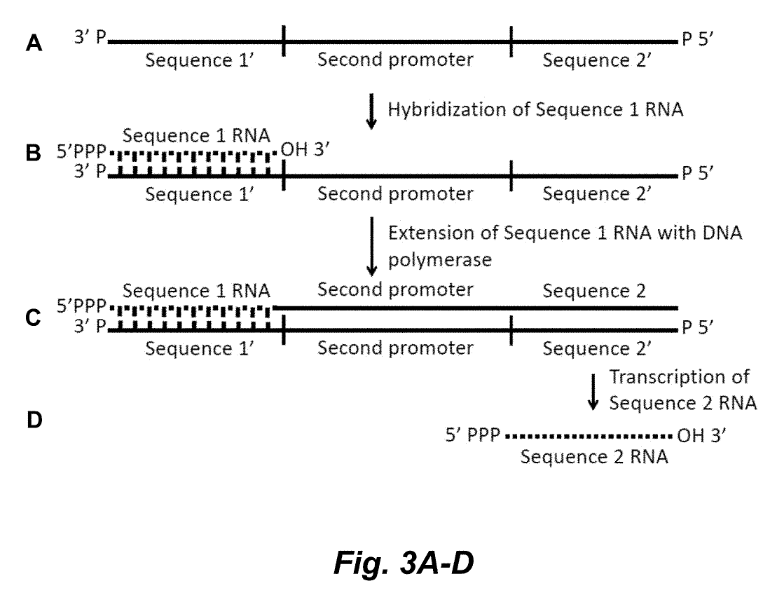

Additionally and optionally, use of one or more translator oligonucleotide with amplifier oligonucleotides allows for a substantial increase in the sensitivity of the system overall, allowing for single nucleic acid molecule detection from a complex mixture of nucleic acid molecules. The structure and function of illustrative amplifier constructs is shown in FIGS. 3-9.

The translator and amplifier constructs described herein are generally synthetic and/or recombinant. The constructs can be comprised wholly of naturally occurring nucleic acids, or in certain embodiments can contain one or more nucleic acid analogues or derivatives. The nucleic acid analogues can include backbone analogues and/or nucleic acid base analogues and/or utilize non-naturally occurring base pairs. Illustrative artificial nucleic acids that can be used in the present constructs include, without limitation, nucleic backbone analogs peptide nucleic acids (PNA), morpholino and locked nucleic acids (LNA), bridged nucleic acids (BNA), glycol nucleic acids (GNA) and threose nucleic acids (TNA). Nucleic acid base analogues that can be used in the present constructs include, without limitation, fluorescent analogs (e.g., 2-aminopurine (2-AP), 3-Methylindole (3-MI), 6-methyl isoxanthoptherin (6-MI), 6-MAP, pyrrolo-dC and derivatives thereof, furan-modified bases, 1,3-Diaza-2-oxophenothiazine (tC), 1,3-diaza-2-oxophenoxazine); non-canonical bases (e.g., inosine, thiouridine, pseudouridine, dihydrouridine, queuosine and wyosine), 2-aminoadenine, thymine analogue 2,4-difluorotoluene (F), adenine analogue 4-methylbenzimidazole (Z), isoguanine, isocytosine; diaminopyrimidine, xanthine, isoquinoline, pyrrolo[2,3-b]pyridine; 2-amino-6-(2-thienyl)purine, pyrrole-2-carbaldehyde, and universal bases (e.g., 2′ deoxyinosine (hypoxanthine deoxynucleotide) derivatives, nitroazole analogues). Non-naturally occurring base pairs that can be used in the present constructs include, without limitation, isoguanine and isocytosine; diaminopyrimidine and xanthine; 2-aminoadenine and thymine; isoquinoline and pyrrolo[2,3-b]pyridine; 2-amino-6-(2-thienyl)purine and pyrrole-2-carbaldehyde; two 2,6-bis(ethylthiomethyl)pyridine (SPy) with a silver ion; pyridine-2,6-dicarboxamide (Dipam) and a mondentate pyridine (Py) with a copper ion.

3. Amplifier Constructs—Structure and Function

a. Structure

Further provided are amplifier DNA constructs. The structure and function of illustrative amplifier constructs is shown in FIGS. 3-9.

In some embodiments, the amplifier DNA construct comprises the following operably linked polynucleotide elements in the 5′ to 3′ direction:

a) a second detection sequence in the antisense orientation;

b) a second RNA polymerase promoter in the antisense orientation; and

c) the first detection sequence in the antisense orientation, as provided in the translator oligonucleotide, as described above and herein. This embodiment is depicted in FIGS. 3-6. The first and second detection sequences can be the same or different.

In some embodiments, the amplifier DNA construct comprises the following operably linked polynucleotide elements in the 5′ to 3′ direction:

a) a second RNA polymerase promoter in the sense orientation;

b) a second detection sequence in the sense orientation;

c) a synthetic transcription termination sequence; and

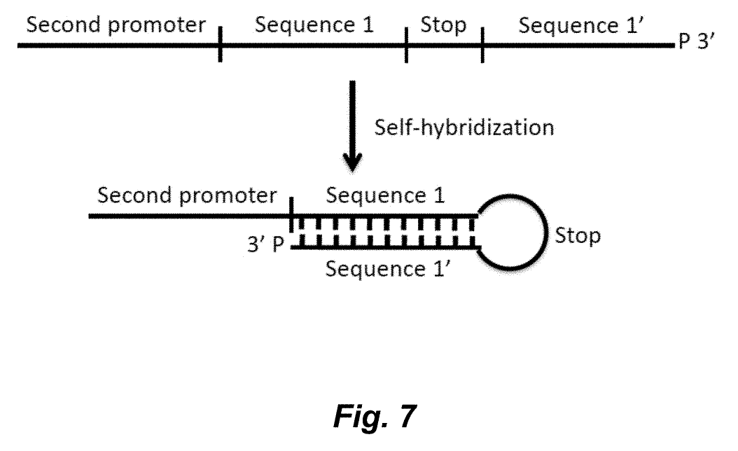

d) the first detection sequence in the antisense orientation, as provided in the translator oligonucleotide, as described above and herein. This embodiment is depicted in FIGS. 7-9. The first and second detection sequences can be the same or different.

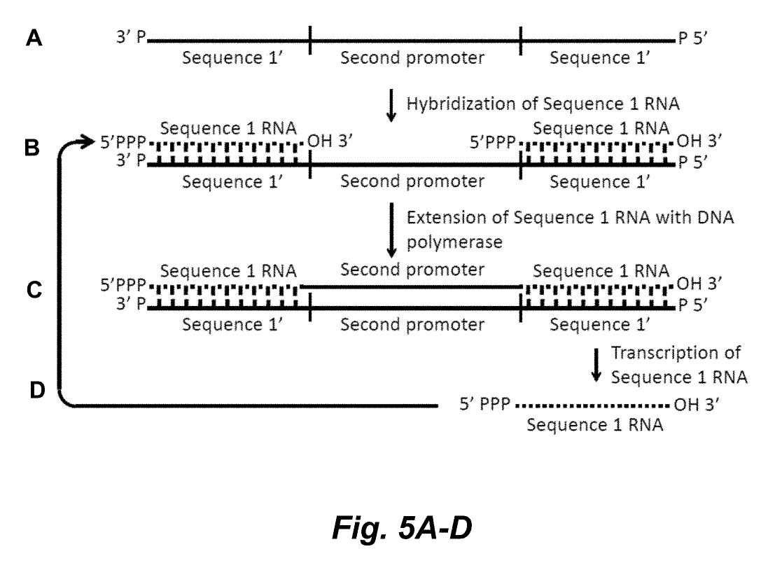

With respect to embodiments of the amplifier DNA constructs, in varying embodiments, the first detection sequence and the second detection sequence are the same. In varying embodiments, the first detection sequence and the second detection sequence are different. In varying embodiments, the RNA oligonucleotide comprising the first detection sequence in the sense orientation produced from the translator DNA construct, as described above and herein, anneals or hybridizes at least to the first detection sequence in the antisense orientation of the amplifier DNA construct, allowing for extension of the RNA oligonucleotide into the transcription termination sequence, the second detection sequence and the promoter in the antisense orientation from the amplifier construct, and further the transcription of the second detection sequence in the sense orientation, producing an RNA oligonucleotide comprising the second detection sequence in the sense orientation. In embodiments where the first detection sequence and the second detection sequence are the same, the RNA oligonucleotide comprising the first detection sequence in the sense orientation produced from the translator DNA construct, as described above and herein, anneals or hybridizes to the first detection sequence and the second detection sequence in the antisense orientation of the amplifier DNA construct (see, FIG. 5). In varying embodiments, the promoter is functional in a prokaryotic cell. In varying embodiments, the first promoter and the second promoter are the same; the promoter in the translator construct and the promoter in the amplifier construct can be the same or different. In varying embodiments, the first promoter and the second promoter are different. In varying embodiments, the promoter functional in a prokaryotic cell comprises a bacteriophage promoter selected from the group consisting of T7, T3 and SP6. In varying embodiments, the amplifier DNA construct has an overall length in the range of about 35 bp to about 85 bp, e.g., from about 38 bp to about 82 bp, e.g., about 35, 40, 45, 50, 55, 60, 65, 70, 75, 80, 85 bp. In varying embodiments, the second detection sequence has a length in the range of about 10 bp to about 50 bp, e.g, from about 10 bp to about 40 bp, e.g., about 10 bp to about 30 bp, e.g., about 10, 15, 20, 25, 30, 35, 40, 45, 50 bp. In varying embodiments, the synthetic transcription termination sequence has a length in the range of about 1 bp to about 5 bp, e.g., about 1, 2, 3, 4, 5 bp.

b. Function

FIG. 3 shows the design for an embodiment of an amplifier oligonucleotide. “Sequence 1 RNA” (e.g., transcribed first detection sequence) produced by the translator oligonucleotide from FIG. 1 or FIG. 2 hybridizes or anneals to the 3′ end of the next generation amplifier oligonucleotide, which provides a primer that can be used by the added DNA polymerase. Extension of this primer by the DNA polymerase through the complement strand of the RNA polymerase promoter activates the promoter for transcription by added RNA polymerase. This leads to the production of “Sequence 2 RNA” (e.g., transcribed second detection sequence) as shown. By a translator DNA construct together with an amplifier DNA construct, substantial increases in signal can be achieved.

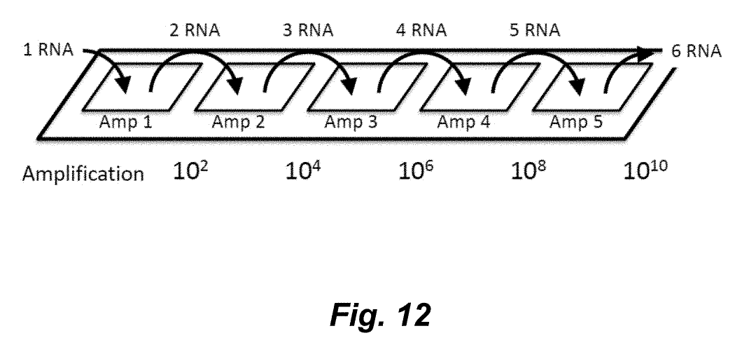

The amplifier DNA constructs depicted in FIGS. 3-6, having the first and second detection sequences in the antisense orientation, do not need to be physically separated from each other, as all of the DNA sequences are of the same strand, that is, there is no possibility of hybridization between successive amplifier oligonucleotides. While segregation of the amplifier oligonucleotides is not necessary, it can facilitate easier detection of the timing of RNA production from each sequence as shown in FIG. 12.

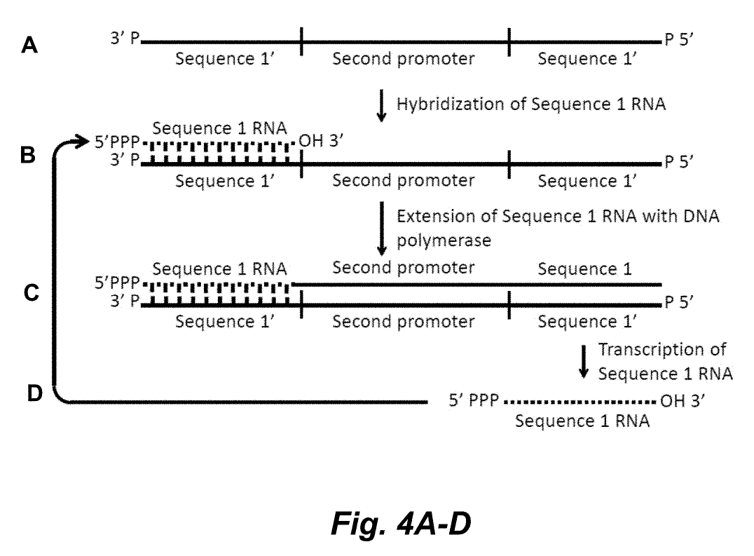

FIG. 4 depicts an embodiment of a cascade amplifier DNA construct, where the first and second detection sequences are the same. The cascade amplifier oligonucleotides depicted in FIGS. 4 and 5 function identically to the amplifier oligonucleotide depicted in FIG. 3, except that the sequences at the beginning and end of the next generation cascade amplifier oligonucleotide are the same. In this way, the transcript produced by the activated cascade amplifier oligonucleotide can itself activate other cascade amplifier oligonucleotides.

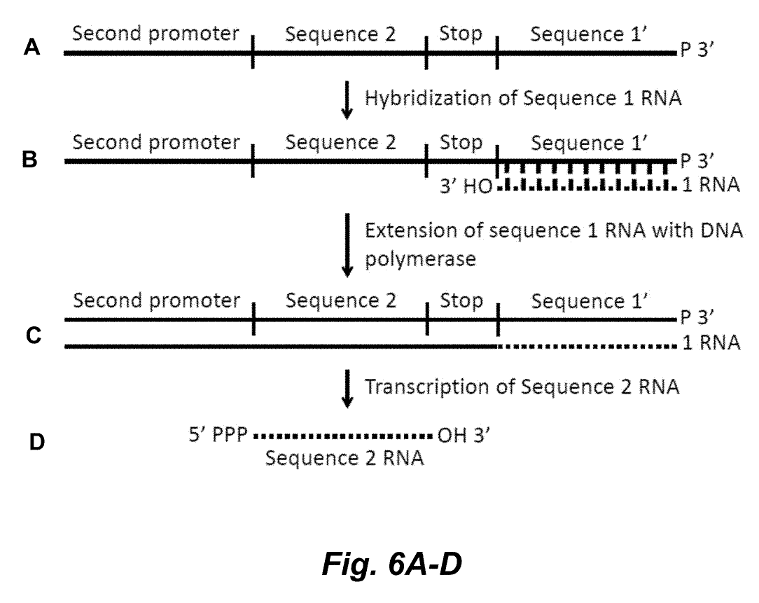

Another embodiment of an amplifier oligonucleotide function is illustrated in FIG. 6 and is as follows. “Sequence 1 RNA” synthesized by the translator oligonucleotide (FIG. 1) hybridizes to the 3′ end of the amplifier oligonucleotide (FIG. 6) at its complement. As appropriate, the hybridization can be in the presence or absence of the mutant RNase H to stabilize the interaction as needed. Added DNA polymerase (e.g., E. coli holoenzyme, large fragment or other DNA polymerase) that can extend a RNA primer on DNA in the presence of all four dNTPs converts the single-stranded amplifier DNA into a double-stranded molecule. This double-stranded DNA can then be transcribed by an RNA polymerase plus CTP, GTP and UTP, excluding A, if using a stop sequence of a short run of dA residues, or all four rNTPs, if using d-isoC or d-isoG as the stop sequence. This will produce new “Sequence 2 RNA” (e.g., transcribed second detection sequence) of known size and with the natural amplification of transcription. By chaining these amplifiers, we can achieve whatever amplification level we desire as shown in FIG. 12.

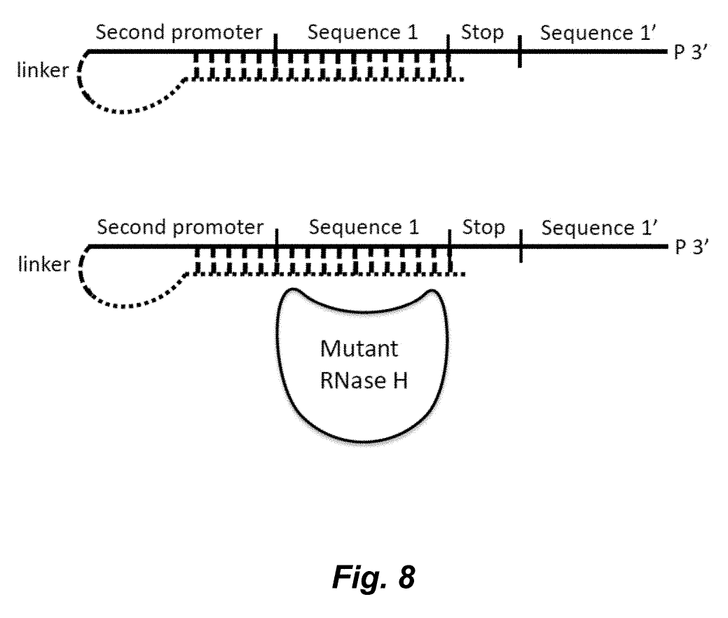

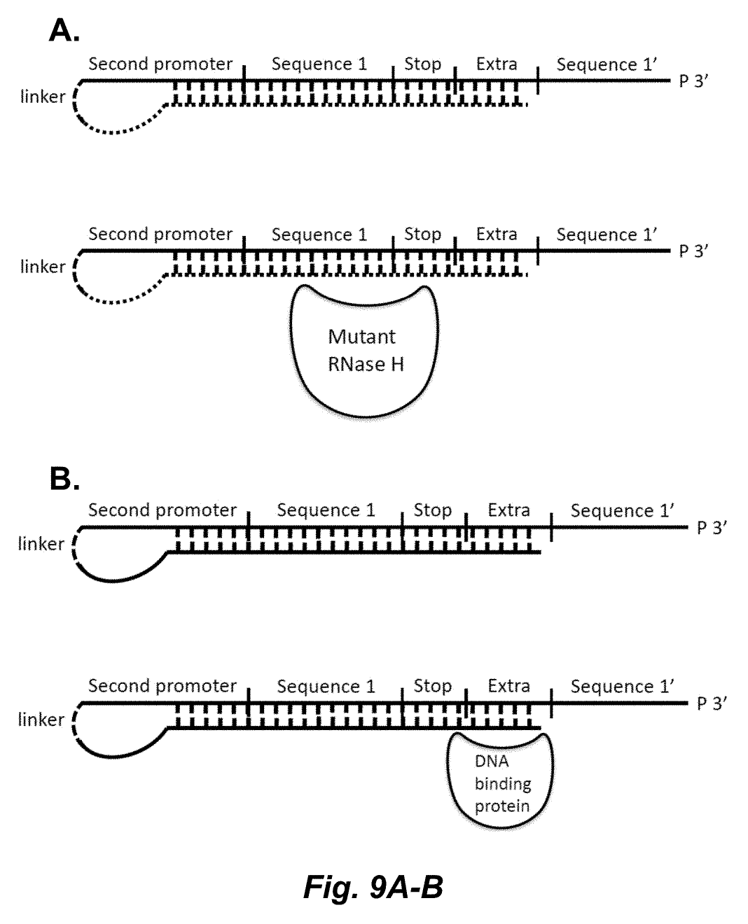

The cascade amplifier oligonucleotide design provided in FIGS. 4-5 does not suffer from the problem of self-complementarity possessed by the cascade amplifier depicted in FIG. 7, wherein the amplifier construction comprises in the 5′ to 3′ direction an RNA polymerase promoter, the first detection sequence in the sense orientation, a synthetic termination sequence, and the first detection sequence in the antisense orientation. The amplifier construct design depicted in FIG. 7 necessitated extra RNA and DNA sequences to reduce or inhibit the formation of internal secondary structures, as shown in FIGS. 8 and 9.

One aspect of the structure of a cascade amplifier oligonucleotide is direct repeats of the sequence 1′ at the beginning and ending of the oligonucleotide (see, FIG. 3). This leads to the possibility of binding of “Sequence 1 RNA” (e.g., transcribed first detection sequence) to both sites as shown in FIG. 5.

The presence of the “Sequence 1 RNA” should not interfere with the function of the RNA polymerase promoter once it is made double stranded. It is possible, depending on the DNA polymerase used that the ultimate structure under the circumstance of two “Sequence 1 RNAs” binding (e.g., as in FIG. 5) will be the same as that seen in FIG. 4, if the DNA polymerase can do nick translation (e.g., an E. coli DNA polymerase holoenzyme), or strand displacement (e.g., a T7 DNA polymerase). In either of these cases, transcription to form new “Sequence 1 RNA” (e.g., transcribed first detection sequence) takes place.

In any of these cases, the addition of mutant RNase H to stabilize the DNA:RNA hybrid can be done, although it may not be necessary as the specific RNA sequences generated, and as a result, their hybridization behavior, can be controlled.

When using single-stranded amplifier oligonucleotides that form internal secondary structures (e.g., the embodiment depicted in FIG. 7), the amplifiers are preferably segregated from each other due to the binding of the amplifier oligonucleotides to each other. For example, in the amplifier embodiment depicted in FIG. 6, the detection sequence (Sequence 2) will be able to bind via its 3′ complementary sequence to the Sequence 2 region of amplifier 1. One strategy to reduce or avoid this undesirable intermolecular hybridization is by attaching the amplifiers to a solid support (e.g., with spatially separated addressable locations) in distinct regions. The RNAs synthesized are free to diffuse between amplifiers as shown in FIG. 12. Assuming a 100× amplification at each transcription step, five amplifier constructs allow for up to a 10¹⁰-fold amplification, bringing a single molecule input into easy detection range.

A cascade amplifier-one capable of amplifying the input RNA itself without the need for serial amplifiers, can be used in conjunction with or as an alternative to a solid support with a plurality of amplifier constructs attached at spatially separated addressable locations. A potential issue could be the formation of internal secondary structures competing for binding of the input “1 RNAs.” The problem is shown in FIG. 7.

In the self-hybridized hairpin depicted in FIG. 7, the 3′ phosphate would prevent extension, but the kinetically favored intramolecular structure formation coupled with the thermodynamically favored formation of the DNA:DNA hybrid over the less favored DNA:RNA hybrid would prevent almost completely the formation of the necessary amplifier DNA:1 RNA hybrid needed to activate the cascade amplifier oligonucleotide. Two possible solutions using an alternative intramolecular RNA and DNA secondary structure are shown in FIGS. 8 and 9, respectively.

The linker in the self-hybridized hairpins depicted in FIGS. 8 and 9 is not absolutely necessary if the kinetics can be driven by high concentrations of the RNA or DNA, or if proteins are used to separate the incorrect secondary structures. Also, the additional sequences within the RNA promoter would be minimized to ensure that no RNA polymerase-mediated transcription would take place without activation after “Sequence 1 RNA” binding and extension. In these cases, a DNA polymerase (e.g., E. coli DNA holoenzyme) which is capable of nick translation would be necessary. Once the nick reaches the region of the RNA promoter that is not hybridized to the RNA or DNA, the DNA polymerase will shift to simple extension of the 3′ end of the DNA, ultimately leading to a fully double stranded RNA polymerase promoter, which will make new “Sequence 1 RNA,” leading to further activation of the cascade amplifier molecules in a feed-forward manner.

Using amplifier oligonucleotides with the same structures as the cascade amplifiers shown in FIGS. 8 and 9 can show superior performance as the double stranded regions should reduce any transiently formed secondary structures that would interfere with binding of the upstream input RNA to the 3′ end of the amplifier oligonucleotide.

The translator and amplifier DNA constructs described herein are generally synthetic and/or recombinant. The constructs can be comprised wholly of naturally occurring nucleic acids, or in certain embodiments can contain one or more nucleic acid analogues or derivatives. The nucleic acid analogues can include backbone analogues and/or nucleic acid base analogues and/or utilize non-naturally occurring base pairs. Illustrative artificial nucleic acids that can be used in the present constructs include, without limitation, nucleic backbone analogs peptide nucleic acids (PNA), morpholino and locked nucleic acids (LNA), bridged nucleic acids (BNA), glycol nucleic acids (GNA) and threose nucleic acids (TNA). Nucleic acid base analogues that can be used in the present constructs include, without limitation, fluorescent analogs (e.g., 2-aminopurine (2-AP), 3-Methylindole (3-MI), 6-methyl isoxanthoptherin (6-MI), 6-MAP, pyrrolo-dC and derivatives thereof, furan-modified bases, 1,3-Diaza-2-oxophenothiazine (tC), 1,3-diaza-2-oxophenoxazine); non-canonical bases (e.g., inosine, thiouridine, pseudouridine, dihydrouridine, queuosine and wyosine), 2-aminoadenine, thymine analogue 2,4-difluorotoluene (F), adenine analogue 4-methylbenzimidazole (Z), isoguanine, isocytosine; diaminopyrimidine, xanthine, isoquinoline, pyrrolo[2,3-b]pyridine; 2-amino-6-(2-thienyl)purine, pyrrole-2-carbaldehyde, and universal bases (e.g., 2′ deoxyinosine (hypoxanthine deoxynucleotide) derivatives, nitroazole analogues). Non-naturally occurring base pairs that can be used in the present constructs include, without limitation, isoguanine and isocytosine; diaminopyrimidine and xanthine; 2-aminoadenine and thymine; isoquinoline and pyrrolo[2,3-b]pyridine; 2-amino-6-(2-thienyl)purine and pyrrole-2-carbaldehyde; two 2,6-bis(ethylthiomethyl)pyridine (SPy) with a silver ion; pyridine-2,6-dicarboxamide (Dipam) and a mondentate pyridine (Py) with a copper ion.

4. Methods of Detecting the Presence of a Target Polynucleotide

Further provided are methods of detecting the presence of a target polynucleotide sequence. In varying embodiments, the target polynucleotide sequence is a single polynucleotide in a complex mixture of polynucleotides, e.g., 1 in 10⁶, 10⁷, 10⁸, 10⁹, 10¹⁰, 10¹¹, 10¹², 10¹³, 10¹⁴, 10¹⁵ polynucleotides.

In some embodiments, the methods comprise:

a) contacting a sample suspected of comprising the target polynucleotide sequence with a translator DNA construct, as described above and herein, under conditions that allow the translator DNA construct to anneal or hybridize to the target polynucleotide; thereby yielding a mixture of annealed or hybridized translator DNA constructs and unannealed or unhybridized translator DNA constructs;

b) separating annealed or hybridized translator DNA construct and target polynucleotide from unannealed or unhybridized translator DNA construct and target polynucleotide;

c) transcribing the first detection sequence, thereby producing an RNA oligonucleotide comprising the first detection sequence in the sense orientation; and

d) detecting the RNA oligonucleotide comprising the first detection sequence in the sense orientation, whereby detecting the RNA oligonucleotide transcribed from the first detection sequence indicates the presence of the target polynucleotide.

In some embodiments, the methods comprise:

a) contacting a sample suspected of comprising the target polynucleotide sequence with a translator DNA construct, as described above and herein, under conditions that allow the translator DNA construct to anneal or hybridize to the target polynucleotide; thereby yielding a mixture of annealed or hybridized translator DNA constructs and unannealed or unhybridized translator DNA constructs;

b) separating annealed or hybridized translator DNA construct and target polynucleotide from unannealed or unhybridized translator DNA construct and target polynucleotide;

c) transcribing the first detection sequence from the translator DNA construct, thereby producing an RNA oligonucleotide comprising the first detection sequence in the sense orientation;

d) contacting the RNA oligonucleotide comprising the first detection sequence in the sense orientation with an amplifier DNA construct, as described above and herein, under conditions that allow the RNA oligonucleotide comprising the first detection sequence in the sense orientation and the amplifier DNA construct to hybridize;

e) extending the RNA oligonucleotide comprising the first detection sequence in the sense orientation with a DNA polymerase thereby producing an extended DNA sequence comprising as operably linked polynucleotide elements in the 5′ to 3′ direction the second promoter and the second detection sequence; and

f) transcribing the second detection sequence from the extended DNA sequence, thereby producing an RNA oligonucleotide comprising the second detection sequence in the sense orientation, whereby detecting the RNA oligonucleotide transcribed from the second detection sequence indicates the presence of the target polynucleotide.

In some embodiments, the target polynucleotide is RNA. In some embodiments, the target polynucleotide is DNA. In varying embodiments, the target polynucleotide is a DNA:RNA hybrid molecule. In some embodiments, the annealed or hybridized translator DNA construct with RNA target polynucleotide is stabilized by a mutated RNase H that does not have nuclease activity. In varying embodiments, one of the translator DNA construct or the target polynucleotide is attached to a solid support. In varying embodiments, the sample is selected from the group consisting of a food sample, a biological sample, a water sample or a soil sample. In varying embodiments, the RNA oligonucleotide is detected by a method selected from the group consisting of radioactive nucleotide incorporation; fluorescent nucleotide incorporation; chemically-derivatized nucleotide incorporation followed by radioactive, fluorescent or enzymatic activity detection; electrochemical detection, molecular conductance detection; detection of the formation of double stranded DNA; detection of transcription of an inherently fluorescent aptamer; detection of transcription of an aptamer that is fluorescent after binding of an otherwise non-fluorescent or minimally-fluorescent molecule; and detection of pyrophosphate produced by transcription and DNA polymerase activity. In varying embodiments, the sample comprises a plurality of different target polynucleotides and a plurality of different translator DNA constructs. In varying embodiments, as few as one target polynucleotide is, e.g., as few as 2, 3, 4, 5, 6, 7, 8, 9, 10, 11, 12, 13, 14, 15, 16, 17, 18, 19, 20 target polynucleotides are, present in the sample.

In some embodiments, the methods entail using a plurality of translator DNA constructs, as described herein, having a plurality of different first detection sequences. In some embodiments, the methods entail using a plurality of amplifier DNA constructs, as described herein, having a plurality of different first detection sequences, which are the same as the plurality of different first detection sequences in the translator DNA constructs. In some embodiments, the methods entail using a plurality of amplifier DNA constructs, as described herein, having a plurality of different second detection sequences.

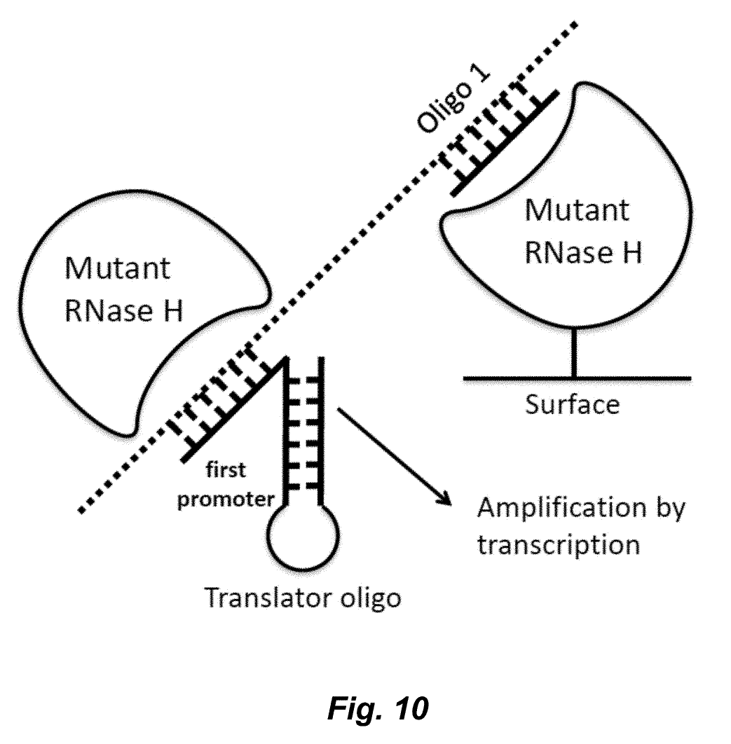

In an illustrative embodiment, FIG. 10 illustrates detection by binding of a translator DNA oligonucleotide after capture of target RNA hybridized to the capture DNA oligonucleotide, including the optional use of an amplifier DNA oligonucleotides to increase the produced signal, and an optional mutant RNase H to stabilize the DNA oligonucleotide:target RNA hybrid.

As depicted in FIG. 10, target RNA or RNAs are recovered from a complex mixture of nucleic acids using a specific capture DNA oligonucleotide. Once separated, a translator DNA oligonucleotide is hybridized to the captured RNA while allowing the capture DNA oligonucleotide:target RNA hybrid to remain bound to the mutant RNase H protein. A second mutant RNase H protein can be added, binding to the site where the translator DNA oligonucleotide is hybridized to the target RNA. Washing removes any unbound translated DNA oligonucleotide. As described above and herein, the structure of the translator DNA oligonucleotide allows it to “translate” binding to a specific nucleic acid sequence to the production of a new nucleic acid sequence that can be detected by a downstream detector.

Any downstream detectors known in the art can be used for detection, including without limitation, chemi-luminescence-based, fluorescence-based, or exploit electrochemical or molecular conductance measurements depending on the application or sensitivity needed. As the sequences produced by either the translator oligonucleotides themselves or by the amplifier oligonucleotides are of known size and sequence, the degradation of the RNA target into the appropriate size for detection is not a concern. Also, as multiple translator oligonucleotides can be associated with a single RNA target, we can require multiple positive signals to validate the presence of the target. These translator oligonucleotides can also be positioned within a short distance of each other (<100 nucleotides), allowing the target RNA to tolerate some degradation during its purification before detection.

5. The Optional Use of Mutated RNase H

Where the target polynucleotide is an RNA or in other instances of the present compositions and methods that involve DNA:RNA hybrids, the DNA:RNA hybrids can be stabilized by use of a ribonuclease H (RNase H). Generally, a mutated RNase H having reduced or eliminated nuclease activity but retaining the ability to bind and stabilize DNA:RNA hybrids is used. Such RNase H mutants are known in the art, and described, e.g., in WO 2004/057016. In varying embodiments, the RNase H is attached, covalently or non-covalently, to a solid support.

In varying embodiments, the compositions and methods described herein employ a mutant RNase H having the substitution of an asparagine (N) for an aspartic acid (D) residue at amino acid position 10 of the protein (D1 ON), as described in Kanaya, et al., J. Biol Chem. (1990) 265(8):4615-21. For convenience of purification, this mutant RNase H protein can be produces as a fusion protein to an expression tag (e.g., maltose binding protein (MBP), His6, glutathione S-transferase, or streptavidin) to facilitate purification. Removal of the expression tag by protease cleavage or the production of the mutant RNase H without fusion to another protein domain is also possible.

DNAs or RNAs of specific sequence bound to a membrane can be detected by exploiting the hybridization of a complementary sequence of either DNA or RNA that has been derivatized in some detectible way, for example, with radioactive nucleotides, fluorescent molecules or enzyme-linkage. Using a similarly derivatized mutant RNase H protein, a DNA of specific sequence can be detected using a complementary RNA hybridized to the DNA molecule without the RNA probe itself being derivatized. Alternatively, a RNA of specific sequence can be detected using a complementary DNA hybridized to the RNA molecule without the DNA itself being derivatized. Both of these detections are mediated by the binding of the mutant RNase H to the DNA:RNA hybrid.

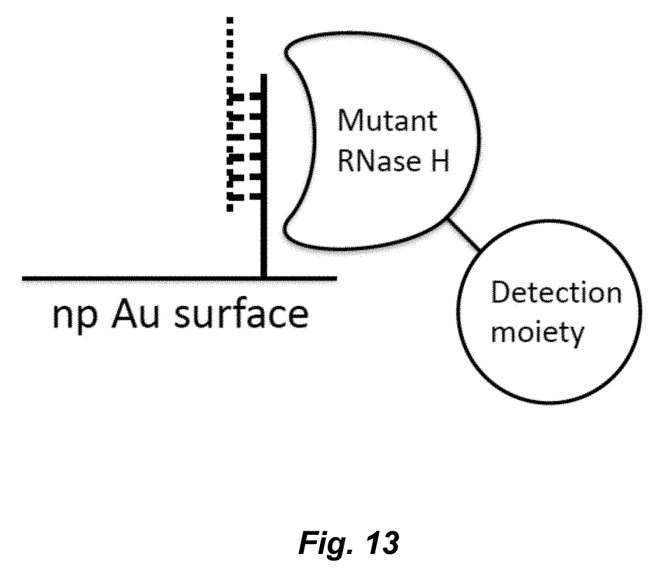

In varying embodiments, a labeled RNase H can be used to detect DNA:RNA hybrids. For example, in some embodiments, binding of an RNA to a probe DNA (e.g., a translator or amplifier DNA construct) bound to a solid support (e.g., a planar or nanoporous gold (npAu) surface) can be detected using a derivatized mutant RNase H protein, as shown in FIG. 13.

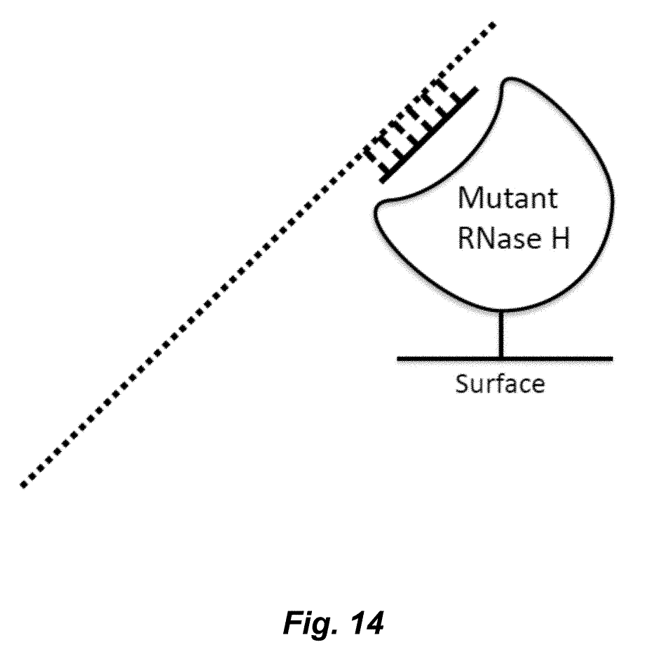

In varying embodiments, the mutant RNase H is attached to a solid support, and can be used to purify and/or isolate a DNA:RNA hybrid from a complex mixture. The mutant RNase H attached to an otherwise separable matrix, for example, a solid support or magnetic bead, can be used to remove any DNA:RNA hybrids from a complex mixture, e.g., left over genomic material, which will not bind to the mutant RNase H protein. The DNA:RNA hybrids can be recovered, substantially increasing the purity of the desired nucleic acids, and reducing the complexity of the nucleic acid sequences present. This embodiment is shown in FIG. 14.

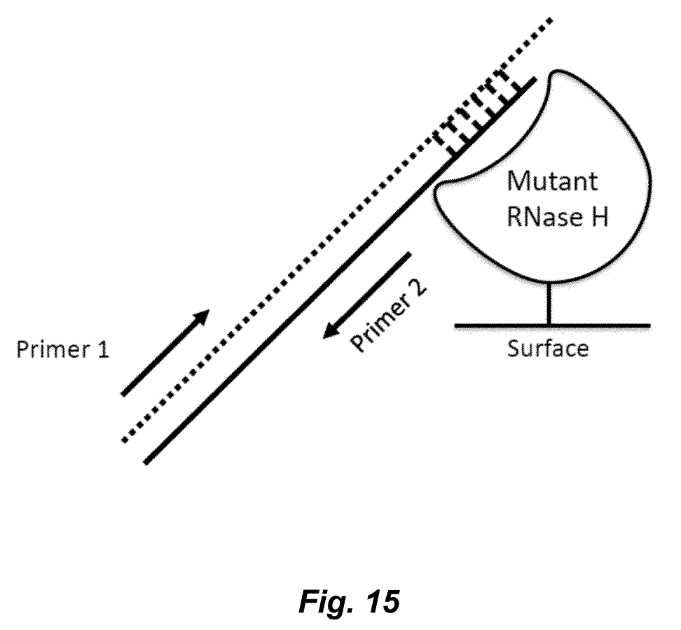

The ability to selective purify RNAs complementary to a specific DNA oligonucleotide can be used as the basis of a detection system. For example, a target RNA or a plurality of target RNAs can be recovered from a complex mixture of nucleic acids using a specific (e.g., complementary) capture DNA oligonucleotide. A mutant RNase H attached to a solid support can be used to purify and/or isolate the DNA:RNA hybrid from the complex mixture. Once the target RNA is separated, reverse transcriptase and dNTPs can be added to synthesize cDNA from the 3′ end of the DNA oligonucleotide. Alternatively, randomized DNA oligonucleotides, for example, random hexamers, or dTn oligonucleotides can be hybridized to the RNA to generate the desired cDNAs. The synthesized cDNA will be amplified either with or without elution from the beads using primers binding 5′ to the site on the RNA where the capture DNA oligonucleotide is bound in the case of where the cDNA is synthesized from the 3′ end of the capture DNA oligonucleotide as shown in FIG. 15 or on either side of the capture DNA oligonucleotide in the case where the cDNA is synthesized from randomized DNA oligonucleotides or dTn oligonucleotides. The advantage of this embodiment is that detection of a target RNA is mediated by sequence recognition at three independent sites (one for the capture DNA oligonucleotide and two for the PCR primer oligonucleotides).

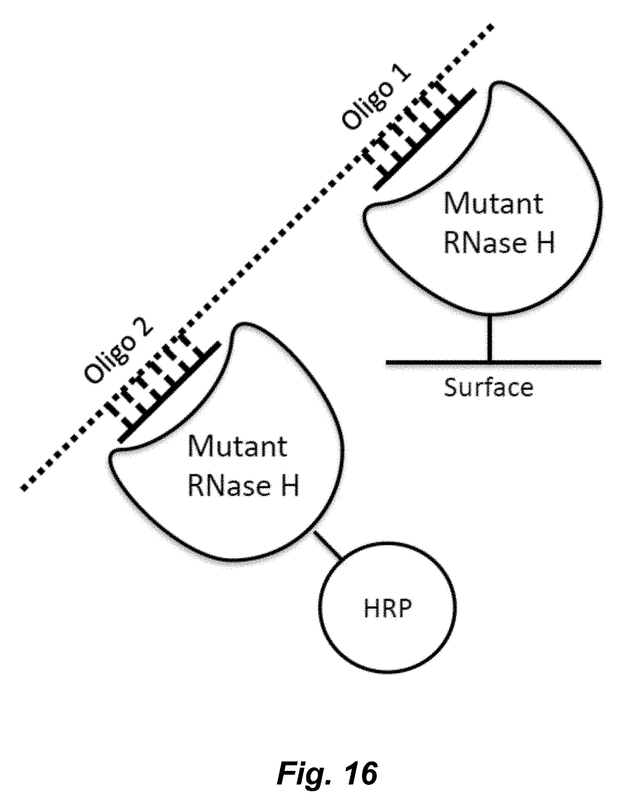

In varying embodiments, a target RNA or plurality of target RNAs are recovered from a complex mixture of nucleic acids using a specific (e.g., complementary) capture DNA oligonucleotide. Once separated, a second DNA oligonucleotide is hybridized to the captured RNA while allowing the capture DNA oligonucleotide:target RNA hybrid to remain bound to the mutant RNase H protein. A second mutant RNase H protein conjugated to a detection moiety, such as HRP, is added, binding to the site where the second DNA oligonucleotide is hybridized to the RNA. Washing removes any unbound, conjugated mutant RNase H, after which the appropriate substrate for enzymatic substrate or other methodology is used to specifically detect the second bound RNase H protein. While this method only has two sequence recognition sites in any given detection (one for the capture DNA oligonucleotide and one for the secondary DNA oligonucleotide), requiring multiple positive sequence recognition events for a specific RNA overcomes this limitation. This embodiment is depicted in FIG. 16.

6. Solid Supports

Further provided are solid supports attached to a plurality of the translator DNA constructs or a plurality of the amplifier DNA constructs, as described herein. As appropriate, attachment can be covalent or non-covalent (e.g., via a hybridizing probe). In varying embodiments, the solid support can be a flat surface (e.g., a chip, a strip, a plate) or a particle (e.g., a bead, a microparticle, a nanoparticle). The solid support can be made of any appropriate material known in the art (e.g., plastic, silica, metal), and commercially available solid supports can be used. In varying embodiments, the solid support is a magnetic bead. As appropriate, a plurality of translator DNA constructs or amplifier DNA constructs can be attached to the solid support at predetermined, spatially separated addressable locations, e.g., as depicted in FIG. 12. Adjacent spatially addressable spaces would be arranged such that detector RNAs produced by one amplifier DNA oligonucleotide would have the shortest diffusible path to reach their cognate amplifier for activation. Detection using any existing detection method, as described above, could be done at each separate spatially addressable space to follow the kinetics of activation to determine the initial concentration of input RNA.

7. Reaction Mixtures

Further provided are reaction mixtures comprising a plurality of the translator DNA constructs, as described herein. Within the plurality of translator DNA constructs, the first detection sequences can all be the same, or the plurality of translator DNA constructs can comprise a plurality of different first detection sequences.

In varying embodiments, the reaction mixture comprises the plurality of translator DNA constructs at a concentration of about 10⁻³ M, about 10⁻⁴ M, about 10⁻⁵ M, about 10⁻⁶ M, about 10⁻⁷ M, about 10⁻⁸ M, about 10⁻⁹ M, about 10⁻¹¹ M, about 10⁻¹² M, or less. In varying embodiment, the molar ratio of the plurality of amplifier DNA constructs to the translator DNA constructs is at least about 2:1, e.g., from about 2:1 to about 10³:1, about 10⁴:1, about 10⁵:1, about 10⁶:1, or more.

In varying embodiments, the reaction mixtures can also comprise a target polynucleotide, e.g., that can hybridize or anneal to the recognition sequence of the translator DNA construct. In varying embodiments, the reaction mixtures can also comprise a test sample suspected of comprising the target polynucleotide. In some embodiments, the reaction mixtures further comprise a plurality of amplifier DNA constructs, as described above and herein. Within the plurality of amplifier DNA constructs, the second detection sequences can all be the same as each other, or the plurality of amplifier DNA constructs can comprise a plurality of different second detection sequences. In some embodiments, the reaction mixtures comprise a plurality of amplifier DNA constructs, as described herein, having a plurality of different first detection sequences, which are the same as the plurality of different first detection sequences in the translator DNA constructs.

As appropriate, the reaction mixtures can also comprise ribo- and deoxyribonucleotides (e.g., rNTPs and dNTPs), RNA and/or DNA polymerases, a mutant RNase H having reduced or eliminated nuclease activity, buffers, detectable label, pH indicators for detection of pH changes indicating polymerization, and other components that can facilitate the desired reaction.

8. Kits

Further provided are kits comprising a plurality of translator DNA constructs, as described herein. Within the plurality of translator DNA constructs, the first detection sequences can all be the same, or the plurality of translator DNA constructs can comprise a plurality of different first detection sequences. In varying embodiments, the plurality of translator DNA constructs can be provided in one or more containers or attached to a solid support, as described herein.

In varying embodiments, the kits can further comprise a plurality of amplifier DNA constructs, as described herein. Within the plurality of amplifier DNA constructs, the second detection sequences can all be the same as each other, or the plurality of amplifier DNA constructs can comprise a plurality of different second detection sequences. In some embodiments, the kits comprise a plurality of amplifier DNA constructs, as described herein, having a plurality of different first detection sequences, which are the same as the plurality of different first detection sequences in the translator DNA constructs. In varying embodiments, the plurality of amplifier DNA constructs can be provided in one or more containers or attached to a solid support, as described herein.

In varying embodiments, the kits can further comprise reagents or components for preparing reaction mixtures, for example, ribo- and deoxyribonucleotides (e.g., rNTPs and dNTPs) (e.g., dNTPs), RNA and/or DNA polymerases, a mutant RNase H having reduced or eliminated nuclease activity, buffers, detectable label, pH indicators for detection of pH changes indicating polymerization, and other components that can facilitate the desired reaction. In some embodiments, the kits comprise control target polynucleotides (e.g., positive and/or negative controls). In varying embodiments, the kits comprise materials for specifically binding the mutant RNase H to a solid support to allow for separation of the bound translator oligonucleotide from the unbound.

### Examples

The following examples are offered to illustrate, but not to limit the claimed invention.

### Example 1: An Illustrative Protocol and Procedure for Detection of an RNA Sequence

Materials

1. A source of RNA containing a sequence to be detected

2. A translator oligonucleotide containing

a. transcriptional hairpin portion, comprising;

i. a first detection sequence in the antisense orientation;

ii. a first RNA polymerase promoter sequence in the antisense orientation;

iii. a hairpin linker;

iv. the first RNA polymerase promoter sequence in the sense orientation; and

v. the first detection sequence in the sense orientation;

b. a flexible linker; and

c. a recognition sequence that hybridizes to a target sequence.

3. An amplifier oligonucleotide containing

a. a second detection sequence in the antisense orientation;

b. a second RNA polymerase promoter in the antisense orientation; and

c. the first detection sequence in the antisense orientation

4. Materials for separating DNA:RNA hybrids from unhybridized DNA and RNA, including but not limited to

a. A mutant RNase H protein containing one or more mutations that prevent RNase H-mediated degradation of the RNA strand of the DNA:RNA hybrid

b. A protein or other moiety for mediating the binding of the mutant RNase H protein to a solid support

c. Reagents for binding and eluting DNA:RNA hybrids from the solid support

5. Reagents for production of RNA and DNA

a. RNA polymerase

b. DNA polymerase

c. rNTPs (rATP, rCTP, rGTP and rUTP)

d. dNTPs (dATP, dCTP, dGTP and dTTP)

e. Buffers and salts appropriate for the enzymes used

Procedure

The sample containing the RNA to be detected is mixed with an excess of translator oligonucleotide. The sample is heated to above the melting temperature of the expected DNA:RNA hybrid and then allowed to cool slowly through the expected melting temperature to facility hybridization.

The hybridized translator oligonucleotide:RNA sample is then applied to the RNase H-binding moiety molecule previously bound to a solid surface, for example, gel beads. The sample is allowed to binding over time, for example, 30 minutes. Longer hybridization times could be used if necessary, for example if the target concentration is low. The DNA:RNA hybrid molecule is then washed free of any non-hybridized translator oligonucleotides or target RNA by taking advantage of the binding of the DNA:RNA hybrids to the solid surface. The volume of wash buffer used would be appropriate to the amount of solid surface, such as gel resin, used. If deemed necessary, the solid support can be used directly for detection or the RNase H-binding moiety can be eluted from the solid support before further detection.

Reagents for production of RNA are added, including but not limited to, rNTPs, buffers and salts, and an RNA polymerase along with reagents for production of DNA, including but not limited to dNTPs, buffers and salts and a DNA polymerase. Accessory proteins can be added if necessary. RNAs produced can then be detected either indirectly, for example by the change in pH that accompanies polymerase action, or directly, by hybridization and further detection, for example using electrochemical detection.

Results

1 mL of a 0.5 OD600 E. coli culture was heated in boiling water to lyse the bacteria, after which the sample was pushed through a 0.2 micron filter by syringe. The liquid collected was used as a simple, but contaminated RNA extraction.

A small amount of this was mixed with the full length translator oligo, which had previously been heated and snap cooled in order to create a double-stranded version with the T7 RNA polymerase promoter.

This mixture was then ramped from 75° C. down to room temperature in order to encourage hybridization.

This solution was diluted in 1 mL of column buffer (20 mM Tris-HCl (pH 7.4), 0.2 M NaCl, 1 mM EDTA) and added to an amylose column that already had the fusion RNase H mutant-maltose binding protein bound to it

The column was washed with 5 mL of column buffer, and the remaining EDTA was washed out with 1×RNA Polymerase Buffer (40 mM Tris-HCl (pH 7.9), 6 mM MgCl₂, 2 mM spermidine, 1 mM dithiothreitol).

The amylose resin was resuspended in 1×RNA Polymerase Buffer, and placed in a separate tube. This was then distributed to four separate tubes, with 500 μL, 50 μL, 5 μL, and 0.5 μL, respectively occupying each tube, and each tube was brought up to the same volume.

[0001]T7 RNA polymerase and rNTPs were added to each tube. They were mixed, and then placed on a 37° C. incubator for 1 hr.

Samples were removed from incubation and half of each was placed in separate tubes.

Klenow fragment, amplifier oligo and dNTPs were added to each of these new tubes. They were mixed, then placed on a 37° C. incubator for 1 hr.

Samples were stored at −20° C. for storage before running on both a 15% TBE and a 15% TBE/7M Urea gel.

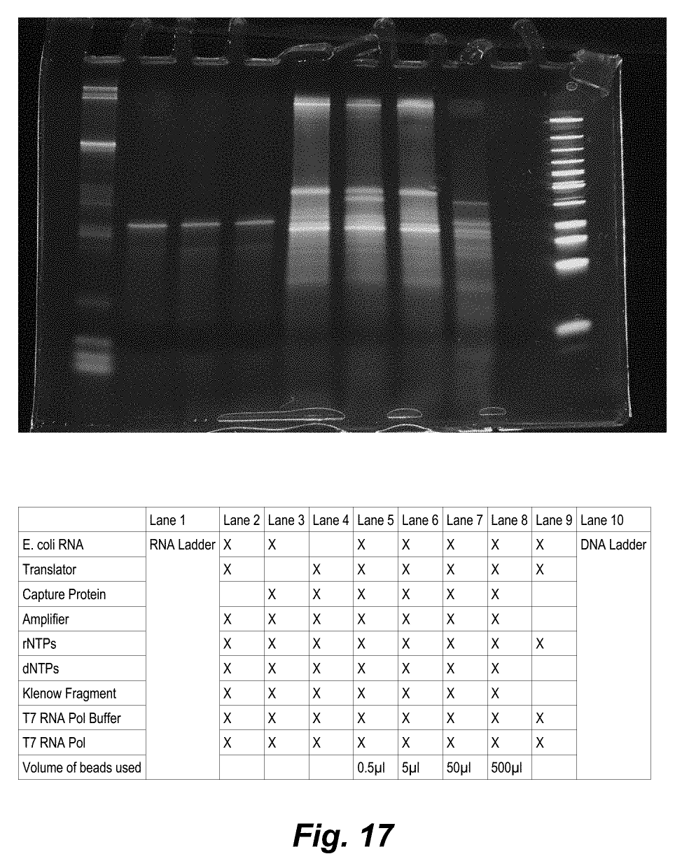

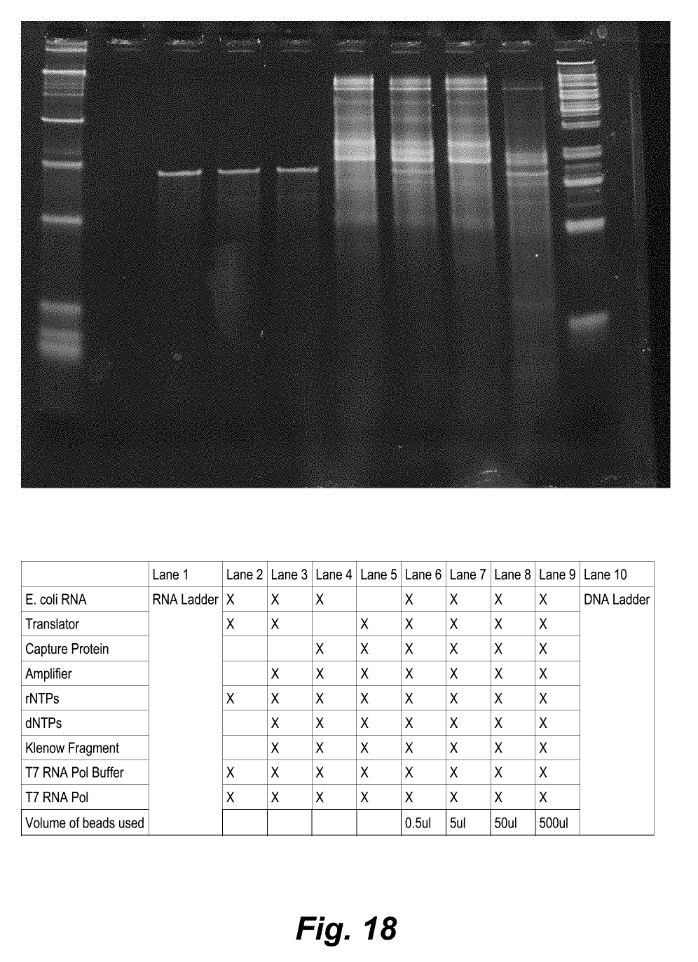

Results are depicted in FIGS. 17 and 18. FIG. 17 depicts the results run in a non-denaturing gel. FIG. 18 depicts the results run in a denaturing gel.

### Example 2: An Illustrative Protocol and Procedure for Activation of an Amplifier Oligonucleotide by a Translator Oligonucleotide

Materials

1. A translator oligonucleotide containing

a. transcriptional hairpin portion, comprising;

i. a first detection sequence in the antisense orientation;

ii. a first RNA polymerase promoter sequence in the antisense orientation;

iii. a hairpin linker;

iv. the first RNA polymerase promoter sequence in the sense orientation; and

v. the first detection sequence in the sense orientation;

b. a flexible linker; and

c. a recognition sequence that hybridizes to a target sequence.

2. An amplifier oligonucleotide containing

a. a second detection sequence in the antisense orientation;

b. a second RNA polymerase promoter in the antisense orientation; and

c. the first detection sequence in the antisense orientation

3. Reagents for production of RNA and DNA

a. RNA polymerase

b. DNA polymerase

c. rNTPs (rATP, rCTP, rGTP and rUTP)

d. dNTPs (dATP, dCTP, dGTP and dTTP)

e. Buffers and salts appropriate for the enzymes used

Procedure

A 10-fold dilution series from 1/10 to 1/10⁴ of the hybridized translator oligonucleotide is made.

The dilution series of the translator oligonucleotide as well as a control with no translator oligonucleotide is added to a constant amount of the appropriate amplifier oligonucleotide having the complementary sequence to the translator oligonucleotide product.

Reagents for production of RNA are added, including but not limited to, rNTPs, buffers and salts, and an RNA polymerase along with reagents for production of DNA, including but not limited to dNTPs, buffers and salts and a DNA polymerase. Accessory proteins can be added if necessary. RNAs produced can then be detected either indirectly, for example by the change in pH that accompanies polymerase action, or directly, by hybridization and further detection, for example using electrochemical detection.

Results

A 10-fold dilution series from 1 μM to 1 nM of the translator oligonucleotide was made. A control containing no translator oligonucleotide was made as well.

One tenth of a reaction volume of each dilution was added to 2 μM amplifier oligonucleotide giving final concentrations of the dilution series of 0.1 μM to 0.1 μM translator oligonucleotide and 0.2 μM amplifier oligonucleotide. The enzyme reaction buffer was added and the sample was preheated to 41° C.

A mix of the appropriate enzymes (DNA and RNA polymerase) and nucleotides (dNTPs and rNTPs) was made and preheated to 41° C.

The oligonucleotide and enzyme mixes were combined and incubated at 41° C. for 1 hour, then allowed to sit at 4° C. overnight.

Samples were mixed with 2×TBE/urea loading dye, heated to 95° C. for 30 seconds before running on a 15% TBE/7M Urea gel.

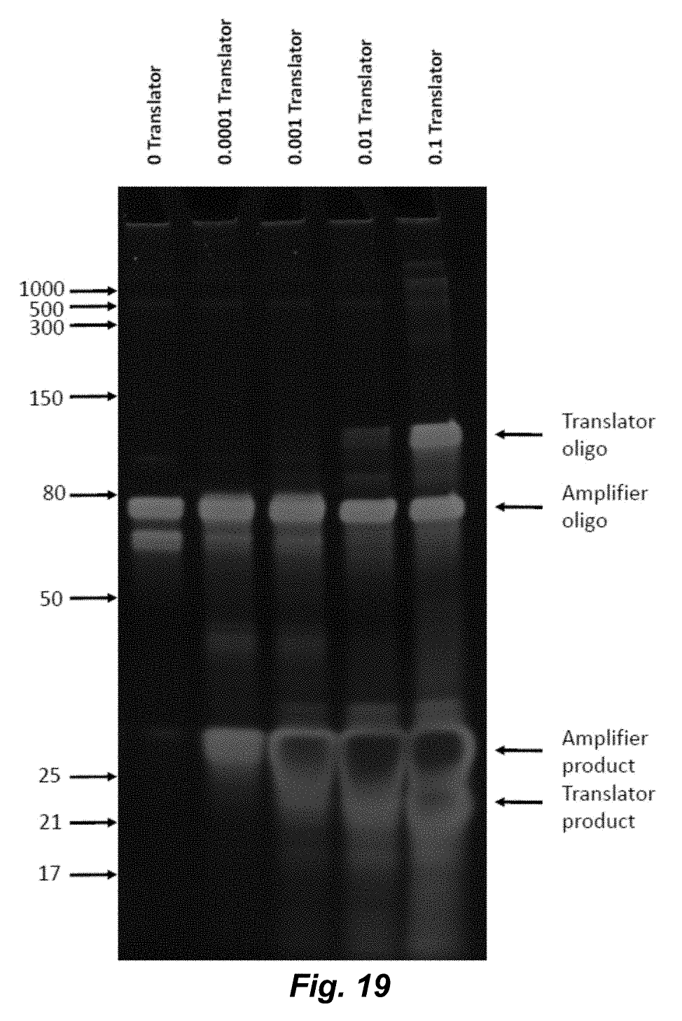

Results are depicted in FIG. 19.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A translator DNA construct comprising the following operably linked polynucleotide elements in the 5′ to 3′ direction:
(a) a transcriptional hairpin portion, comprising:
(i) a first detection sequence in the antisense orientation;
(ii) a first RNA polymerase promoter sequence in the antisense orientation;
(iii) a hairpin linker;
(iv) the first RNA polymerase promoter sequence in the sense orientation; and
(v) the first detection sequence in the sense orientation;
(b) a flexible linker; and
(c) a recognition sequence that hybridizes to a target sequence.

2. (canceled)

3. The translator DNA construct of claim 1, wherein the first detection sequence in the sense orientation can be transcribed into RNA from the promoter, producing an RNA oligonucleotide comprising the first detection sequence in the sense orientation.

4. The translator DNA construct of claim 1, wherein the first promoter is functional in a prokaryotic cell comprising a bacteriophage promoter selected from the group consisting of T7, T3 and SP6.

5-9. (canceled)

10. The translator DNA construct of claim 1, wherein the hairpin linker is a linker selected from the group of linkers consisting of a polyethylene glycol linker and a deoxyribose phosphodiester linker without nucleotide bases.

11. (canceled)

12. The translator DNA construct of claim 1, wherein the flexible linker is a linker selected from the group of linkers consisting of a polyethylene glycol linker and a deoxyribose phosphodiester linker without nucleotide bases.

13. (canceled)

14. The translator DNA construct of claim 1, wherein the translator DNA construct is attached to a solid support.

15. The translator DNA construct of claim 1, further comprising an amplifier DNA construct comprising the following operably linked polynucleotide elements in the 5′ to 3′ direction:
(a) a second detection sequence in the antisense orientation;
(b) a second RNA polymerase promoter in the antisense orientation; and
(c) a second copy of the first detection sequence in the antisense orientation.

16. The amplifier DNA construct of claim 15, wherein the first detection sequence and the second detection sequence are the same.

17-19. (canceled)

20. The amplifier DNA construct of claim 15, wherein the second promoter functional in a prokaryotic cell comprises a bacteriophage promoter selected from the group consisting of T7, T3 and SP6.

21. The amplifier DNA construct of claim 15, wherein the first promoter and the second promoter are the same.

22-24. (canceled)

25. An amplifier DNA construct comprising the following operably linked polynucleotide elements in the 5′ to 3′ direction:
(a) a second RNA polymerase promoter in the sense orientation;
(b) a second detection sequence in the sense orientation;
(c) a synthetic transcription termination sequence; and
(d) the first detection sequence in the antisense orientation, as in claim 1.

26. The amplifier DNA construct of claim 25, wherein the first detection sequence and the second detection sequence are the same.

27. (canceled)

28. The amplifier DNA construct of claim 25, wherein the RNA oligonucleotide comprising the first detection sequence in the sense orientation produced from the translator DNA construct of any one of claims 1 to 12 anneals or hybridizes to the first detection sequence in the antisense orientation of the amplifier DNA construct, allowing for extension of the RNA oligonucleotide into the transcription termination sequence, the second detection sequence and the promoter in the antisense orientation from the amplifier construct, and further the transcription of the second detection sequence in the sense orientation, producing an RNA oligonucleotide comprising the second detection sequence in the sense orientation.

29. The amplifier DNA construct of claim 25, wherein the promoter is functional in a prokaryotic cell comprising a bacteriophage promoter selected from the group consisting of T7, T3 and SP6.

30. The amplifier DNA construct of claim 25, wherein the first promoter and the second promoter are the same.

31.-46. (canceled)

47. A method of detecting the presence of a target polynucleotide sequence comprising:
(a) contacting a sample suspected of comprising the target polynucleotide sequence with a translator DNA construct of claim 1 under conditions that allow the translator DNA construct to anneal or hybridize to the target polynucleotide; thereby yielding a mixture of annealed or hybridized translator DNA constructs and unannealed or unhybridized translator DNA constructs;
(b) separating annealed or hybridized translator DNA construct and target polynucleotide from unannealed or unhybridized translator DNA construct and target polynucleotide;
(c) transcribing the first detection sequence, thereby producing an RNA oligonucleotide comprising the first detection sequence in the sense orientation; and
(d) detecting the RNA oligonucleotide comprising the first detection sequence in the sense orientation, whereby detecting the RNA oligonucleotide transcribed from the first detection sequence indicates the presence of the target polynucleotide.

48. The method of claim 47, further comprising:
(a) contacting the RNA oligonucleotide comprising the first detection sequence in the sense orientation with an amplifier DNA construct of claim 25 under conditions that allow the RNA oligonucleotide comprising the first detection sequence in the sense orientation and the amplifier DNA construct to hybridize;
(b) extending the RNA oligonucleotide comprising the first detection sequence in the sense orientation with a DNA polymerase thereby producing an extended DNA sequence comprising as operably linked polynucleotide elements in the 5′ to 3′ direction the second promoter and the second detection sequence; and
(c) transcribing the second detection sequence from the extended DNA sequence, thereby producing an RNA oligonucleotide comprising the second detection sequence in the sense orientation, whereby detecting the RNA oligonucleotide transcribed from the second detection sequence indicates the presence of the target polynucleotide.

49. The method of claim 47, wherein the target is selected from the group of targets consisting of: a polynucleotide is RNA, DNA and a DNA:RNA hybrid molecule.

50-51. (canceled)

52. The method of claim 47, wherein the annealed or hybridized translator DNA construct with RNA target polynucleotide is stabilized by a mutated RNase H that does not have nuclease activity.

53-54. (canceled)

55. The method of claim 47, wherein the RNA oligonucleotide is detected by a method selected from the group consisting of radioactive nucleotide incorporation; fluorescent nucleotide incorporation; chemically-derivatized nucleotide incorporation followed by radioactive, fluorescent or enzymatic activity detection; electrochemical detection, molecular conductance detection; detection of the formation of double stranded DNA; and detection of pyrophosphate produced by transcription and DNA polymerase activity.

56-57. (canceled)
